(19) United States
(12) Patent Application Publication
(10) Pub. No.: US 2013/0066660 A1
(43) Pub. Date: Mar. 14, 2013

(54) EVENT RESERVATION SYSTEM

(71) Applicants: Jim Kopitzke, Oak Park, CA (US); Robin Barber, Palmdale, CA (US)

(72) Inventors: Jim Kopitzke, Oak Park, CA (US); Robin Barber, Palmdale, CA (US)

(73) Assignee: PRECISION DYNAMICS CORPORATION, Valencia, CA (US)

(21) Appl. No.: 13/608,056

(22) Filed: Sep. 10, 2012

Related U.S. Application Data
(60) Provisional application No. 61/532,749, filed on Sep. 9, 2011.

(51) Int. Cl. G06Q 10/02

(52) U.S. Cl. 705/5

(57) ABSTRACT

A pre-event admission and purchasing system is disclosed in which a patron, patient or the like enters the required personal information, financial transaction information, and makes advance purchases before admission to a venue, healthcare facility or the like. An RFID wristband having this information stored thereon or linked to a database via a human readable or machine readable code tracks such admissions, purchases, access and the like during the stay. The database or master index for the patrons, patients or other individuals captures pertinent data and links that data needed by the various applications or systems involved in the event.

### BACKGROUND OF THE INVENTION

The present invention generally relates to ticketing systems. More particularly, the present invention resides in a ticketing system which integrates pre-event admission selections and fees with an RFID wristband.

There exist many different types of events and venues which provide leisure and entertainment. Many of such events and venues are temporary or seasonal in nature. For example, such venues and events may only occur during the warmer spring and summer months of the year, while the facility is closed during the winter. Such venues, for example, may include seasonal water parks, smaller theme parks, traveling theme parks, music venues, outdoor theaters, festivals, sporting events and the like. Oftentimes, these venues or events not only require a ticket for admittance, but also parking, concessions within the venue, optional activities within the event or venue and the like. Because of the many attractions in the venue, each of these has separate admission or usage fees, and separate transactions must be made to pay for each. This requires that the patrons carry a substantial amount of cash to pay for parking, tickets, concessions, other usage fees, etc. as well as the employees of the venue having appropriate change. Alternatively, the venue will need to be equipped with the appropriate communications and electronic devices to accept credit card payments and the like. In any event, this can cause long lines and waits for the patrons and increases the costs and complexity of running the event or venue.

Entertainment venues and events often have a number of challenges with staffing. Due to the seasonal nature of the venues and the tight profit margins, such venues and events often hire very young people to staff gate and sales positions. Hiring the additional employees is expensive for the venue or event. Moreover, due to the nature of the employees, they are inexperienced and occasionally steal from the cash receipts.

In some venues and events, self-service kiosks are implemented. The patrons can purchase the tickets for admission, etc. at the kiosk at the event. While removing the need for as many employees for the venue or event, such kiosks oftentimes create long lines due to the amount of information which must be input by each patron as well as consummating the financial transaction at the kiosk.

Accordingly, an improved event and venue ticketing system is needed which overcomes the disadvantages with existing systems described above. The present invention fulfills these needs and provides other related advantages.

### SUMMARY OF THE INVENTION

The present invention is direct to a computer-based method performed by an event reservation system comprising a web portal, a kiosk and at least one event scanner. The method comprises steps performed through the web portal, at the kiosk, or at a site of the event reservation. Through the web portal, the system receives an order from a user for an event reservation, associates a transaction code with the order and a patron ID with the user, and provides the transaction code and patron ID to the user. At the kiosk, the system receiving the transaction code or patron ID from the user, and dispenses an identification article bearing a machine readable code corresponding to the transaction code or patron ID. At a site of the event reservation, the system reads the machine readable code from the identification article, and permits the user benefits of the event reservation in response to reading the machine readable code.

In this computer based method, the event reservation may comprise room accommodations. In this instance, the method determines whether a current time has reached a check-in time for the room accommodations, and provides availability and location information for the room accommodations in response to reading the machine readable code if the current time has reached the check-in time.

The computer based method also receives discretionary monetary funds from the user, establishes an electronic spending account associated with the transaction code or patron ID and containing the discretionary monetary funds, and permits the user to make purchases with the discretionary monetary funds in the electronic spending account in response to reading the machine readable code.

The computer based method also offers the user through the web portal a registration page for a social media website account, receives account information from the user corresponding to the social media website account, and associates the social media website account with the machine readable code. The order is associated with the social media website account of the user, and pushes an information post to the social media website account in response to reading the machine readable code. A social media website link is offered to the user in response to the step of receiving the order, and the system receives a response from the user that authorizes pushing information posts to the social media website account.

In an alternate embodiment, the event reservation process involves receiving an order for an event reservation from a user through a web portal into an electronic database. The order is processed to associate a transaction code with the order and a patron ID with the user. The transaction code and patron ID are provided to the user through the web portal. The transaction code or patron ID from the user are input at a kiosk which then dispenses an identification article bearing a machine readable code associated with the event reservation ordered by the user through the web portal. At the site of the event reservation, the machine readable code is read from the identification article. Assuming that the transaction code and/or patron ID or authentic, the user is permitted to redeem the order received through the web portal.

Where the event reservation is for room accommodations, the process determines whether a current time has reached a check-in time for the room accommodations, and provides availability and location information for the room accommodations in response to reading the machine readable code at the site of the event reservation if the current time has reached the check-in time.

The process may involve receiving discretionary monetary funds from the user and establishing an electronic spending account associated with the transaction code or patron ID that contains the discretionary monetary funds. The user is then permitted to make purchases with the discretionary monetary funds in the electronic spending account in response to reading the machine readable code at the site of the event reservation.

The user is offered through the web portal a registration page for a social media website account. The database receives account information from the user corresponding to the social media website account, and associates the social media website account with the machine readable code. The order is associated with the social media website account of the user, and information posts may be pushed to the social media website account in response to reading the machine readable code at the site of the event reservation. A social media website link may be offered to the user in response to the step of receiving the order, and a response may be received from the user that authorizes pushing information posts to the social media website account.

An event reservation and redemption process involves inputting an order for an event reservation from a user into a database, associating a transaction code with the order and a patron ID with the user, providing the transaction code and/or patron ID to the user, dispensing an identification article bearing a machine readable code corresponding to the transaction code or patron ID, and electronically reading the machine readable code from the identification article to allow the user to utilize the benefits of the event reservation. Where the event reservation comprises room accommodations, the process involves determining whether a current time has reached a check-in time for the room accommodations, and providing availability and location information for the room accommodations in response to electronically reading the machine readable code at a site of the event reservation if the current time has reached the check-in time.

The event reservation and redemption process further involves receiving discretionary monetary funds from the user, establishing an electronic spending account associated with the transaction code or patron ID and containing the discretionary monetary funds, and permitting the user to make purchases with the discretionary monetary funds in the electronic spending account in response to electronically reading the machine readable code at a site of the event reservation.

The user may be offered a registration page for a social media website account, account information may be received from the user corresponding to the social media website account, and the social media website account may be associated with the machine readable code. The order may be associated with the social media website account of the user, and an information post may be pushed to the social media website account in response to electronically reading the machine readable code at a site of the event reservation.

An event reservation system has a database and data processor configured for receiving an order from a user for an event reservation, associating a transaction code with the order, and associating a patron ID with the user. A kiosk is in electronic communication with the database and data processor, and is configured to receive the transaction code or patron ID from the user and to dispense an identification article bearing a machine readable code corresponding to the transaction code or patron ID. An event machine is in electronic communication with the database and data processor. The event machine is configured to read the machine readable code from the identification article and to permit the user to exercise benefits associated with the event reservation.

The event reservation preferably comprises room accommodations, park admissions, parking reservations, locker rentals, stroller rentals, wheelchair rentals, and/or VIP privileges. Where the event reservation comprises room accommodations, the event machine is configured to determine whether a current time has reached a check-in time for the room accommodations, and to provide availability and location information for the room accommodations in response to reading the machine readable code if the current time has reached the check-in time.

The event reservation system also includes an electronic spending account maintained in the database and in electronic communication with the data processor. The electronic spending account is associated with the transaction code or the patron ID. The electronic spending account has a balance value commensurate with discretionary monetary funds deposited by the user. The event machine is configured to permit the user to make purchases with the discretionary monetary funds in the electronic spending account in response to reading the machine readable code.

A social media website link is included in the database and is configured to receive account information from the user corresponding to a social media website account of the user. The link associates the social media website account with the machine readable code from the identification article. The social media website link is configured to associate the order with the social media website account of the user and to push an information post to the social media website account of the user in response to the event machine reading the machine readable code from the identification article.

The identification article preferably comprises a wristband, a label, an ID card, or a ticket bearing a bar code or an RFID chip. The wristband comprises an RFID inlay and/or a printed barcode linked to the transaction code, the patron ID and/or a pre-funded spending account.

The order comprises patron information, purchase information, social media authorizations, and electronic spending account information. The patron information comprises name, address and payment information. The purchase information comprises admission tickets, rental items, VIP privileges, and parking passes. The rental items comprise lockers, strollers, and wheelchairs. The database and data processor are integrated with third-party systems providing event ticketing, point-of-sale/payment processing, social media providers, parking management, and/or rental locker management.

A transaction receipt is provided to the user, wherein the transaction receipt contains the transaction code, the patron ID, a confirmation/transaction number and/or a purchase summary. The transaction code and/or patron ID corresponds to access rights, privileges, authorization and/or rentals of the user. The identification article may be mailed to the user or placed on will-call at an event site.

The kiosk is configured to receive the transaction code or patron ID by scanning a printed barcode or manual entry of the transaction code and/or patron ID via keypad or touchscreen. The kiosk may also be configured to receive a new order from the user for a new event reservation. In various embodiments, the kiosk may be positioned in different locations. The kiosk may be positioned at an entry point to the event and be configured to grant or deny entry to or privileges of the event based upon the transaction code and/or patron ID when the user presents the identification article to an RFID reader. In this embodiment, the kiosk may comprise a gate, portal, turnstile, or mobile device.

The kiosk may be positioned at a rental location in the event and be configured to release rental items or permit access to lockers based upon the transaction code and/or patron ID when the user presents the identification article to an RFID reader. In this embodiment, the kiosk may comprise a fixed or mobile device. The kiosk may be positioned at a point-of-sale outlet in the event and be configured to grant or deny a point-of-sale transaction based upon a pre-funded spending account and the transaction code and/or patron ID when the user presents the identification article to an RFID reader.

In this embodiment, the kiosk may comprise a fixed or mobile device. A plurality of kiosks may be positioned through the event and be configured to push an information post to the user's social media website account based upon the transaction code and/or patron ID when the user presents the identification article to an RFID reader. In this embodiment, the kiosk may again comprise a fixed or mobile device.

The event reservation may correspond to a healthcare appointment, multi-media purchases, menu selections, discharge instructions and/or infection surveillance. The multi-media purchases may comprise television and/or movies. In this embodiment, the database and data processor are integrated with third-party systems comprising a visitor registration system, a patient database, an access control system, a hospital multi-media system, a patient billing service, a food service, and/or a patient/staff location system. The user may access the database and data processor over the internet through a personal computer, tablet computing device, or smart phone.

Other features and advantages of the present invention will become apparent from the following more detailed description, taken in conjunction with the accompanying drawings, which illustrate, by way of example, the principles of the invention.

### BRIEF DESCRIPTION OF THE DRAWINGS

The accompanying drawings illustrate the invention. In such drawings:

FIG. 1 is a flow chart illustrating an embodiment of a pre-event/admission method of the present invention;

FIG. 2 is a flow chart illustrating an embodiment of an event site method of the present invention;

FIG. 3 is a flow chart illustrating an embodiment of database interaction of the present invention;

FIG. 4 is a flow chart illustrating an embodiment of a kiosk method in a healthcare setting;

FIG. 5 is a flow chart illustrating an embodiment of an overall computer system of the present invention;

FIG. 6 is a flow chart illustrating an embodiment of an online reservations method of the present invention;

FIG. 7 is a flow chart illustrating an embodiment of a method for interacting with a kiosk of the present invention; and

FIG. 8 is a flow chart illustrating an embodiment of a method of an information query through a kiosk of the present invention.

### DETAILED DESCRIPTION OF THE INVENTION

As shown in the accompanying drawings, for purposes of illustration, the present invention is directed to a pre-event admission process, i.e., event reservation/ticketing system. The system of the present invention is configured to integrate the various fees and services of the event or venue using an identification article, i.e., barcode or RFID wristbands. The identification article is used to link the patron with the services that they have ordered.

The term “event reservation” is intended to encompass various services, including admission, parking, rentals, VIP privileges, and other similar services as one may find at amusement parks, stadiums, arenas, rides, healthcare facilities, or any other similar facility or service. “Rentals” refers to equipment such as lockers, strollers, wheelchairs, etc. “VIP privileges” refers to front-of-line, limited area access, etc.

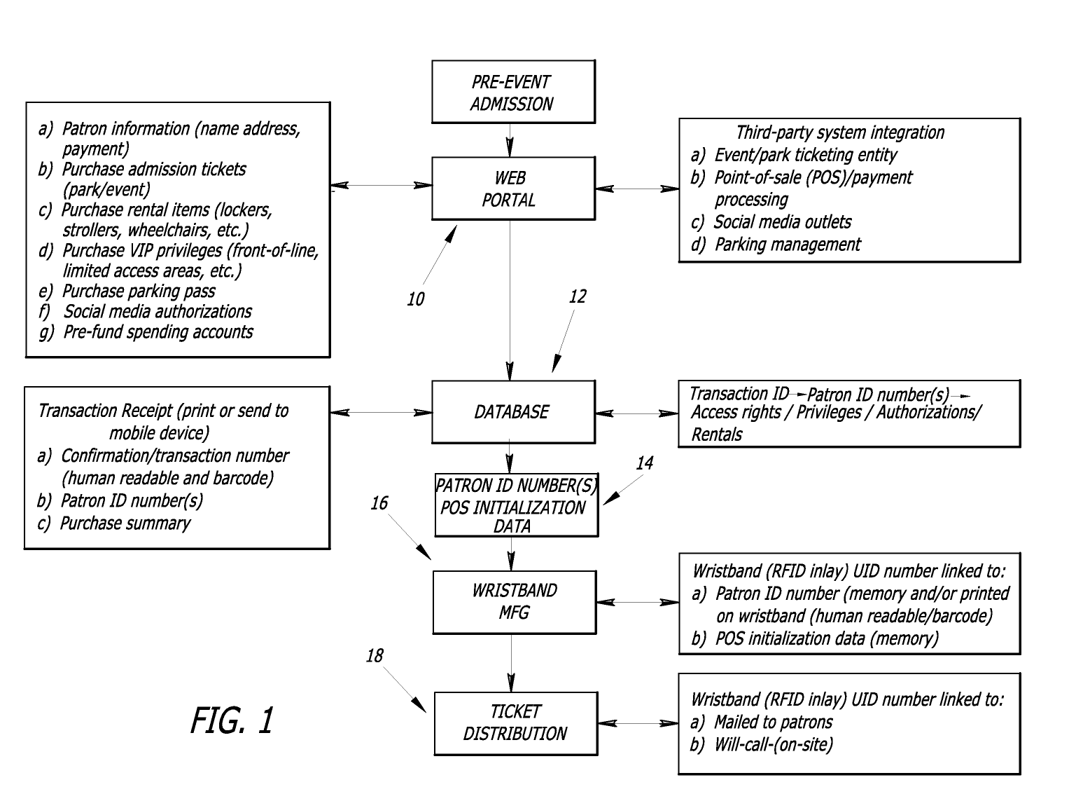

With reference now to FIG. 1, the present invention provides a web portal 10 as a component of the pre-event/admission process. Through the web portal 10, the patron can input his or her information, such as name, address, payment information and the like. The patron may purchase admission tickets through the web portal, as well as additional items, such as parking, rental items (lockers, strollers, wheelchairs, etc.). In some instances the patron may have the opportunity of purchasing VIP privileges (front-of-line, limited access areas, and the like). The patron may also pre-fund a spending account, for point-of-sale payment processing such as concessions, etc. In one embodiment of the present invention, the patron may also authorize social media interaction and automatic push messages, and the like.

By means of the web portal 10, the system integrates the event and parking ticketing entity, the point-of-sale and payment processing, parking management, as well as social media outlets. Some or all of these features may be provided by the event or venue, while others (such as the social media outlets) may be accessed, monitored or controlled by third parties. In any event, these pre-authorizations, sales, etc. are integrated with one another.

With continuing reference to FIG. 1, this information is saved within a database 12. A transaction identification is associated with each patron identification, and these identifications are linked to the patron's access rights, privileges, authorizations, rentals, etc.

Typically, the patron is provided a transaction receipt 14. This may be a printable transaction receipt or an electronic receipt which may be sent to the user's computer, telephone, or other mobile device. Typically, the transaction receipt will include a confirmation/transaction number which may be human and/or machine readable, such as containing a barcode or other electronically readable symbol. Typically, the transaction receipt will include the patron's identification number as well as a summary of the purchases made.

In one embodiment, a wristband is manufactured 16. The wristband preferably has an RFID inlay with a unique identification code or number (UID) which is linked to the patron's identification number and the point-of-sale initialization data. The wristband may also or alternatively bear a barcode or similar encoding structure. The patron identification number may be placed within the RFID's memory, and/or printed on the wristband in human readable and/or machine readable (such as barcode) fashion. The point-of-sale initialization data may be stored in the RFID's memory, or the patron ID number and point-of-sale initialization data may be linked to the database 12 and updated as needed.

When a specific wristband is manufactured as part of the pre-event admission process, as illustrated in FIG. 1, the ticket is distributed 18, in the form of a wristband with a unique identification as an element of RFID inlay, which is either mailed to the patron or placed at will-call on site at the venue or event for pickup by the patron when attending the event. The wristband may also be dispensed by a kiosk or similar device once the patron authenticates his/her identity for the kiosk, s described below. The UID number of the inlay manufactured in the wristband is the link between the patron and the database information.

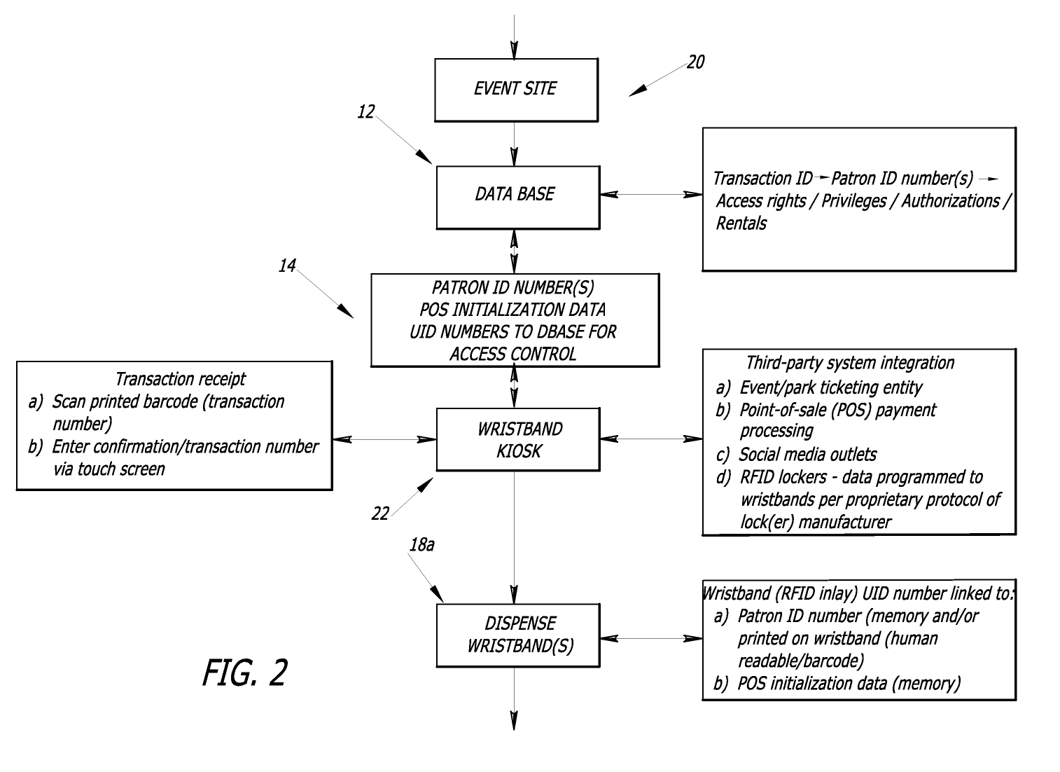

With reference now to FIG. 2, the steps of FIG. 1 including the web portal 10 interaction and access, and the database 12 file creation and storage have already occurred, such that there is a transaction identification correlating with a patron identification number or code or name, etc., which is connected to the access rights, privileges, authorizations, rentals, etc. that the patron has selected and paid for. However, in this case, at the event site or venue 20, the patron approaches a kiosk 22 and scans the machine readable code, such as a barcode, at the kiosk or enters the confirmation/transaction number or code via a keypad, touchscreen or the like. A wristband is dispensed by the kiosk 18a which has the preselected and pre-purchased events, services, etc. either stored in the memory of the wristband or the wristband contains a human and/or machine readable code or symbol which links the wristband to the database where this information is stored. For example, the UID number or code of the inlay manufactured RFID wristband may be the link between the patron and the database information.

In one embodiment, the kiosk may also be set up and designed so as to handle a new transaction as outlined in FIG. 1. In such an instance, the patron enters his information, payment information, makes the relevant purchase of tickets, rental items, etc. at the kiosk instead of doing so through the web portal before the event. A kiosk as described herein is a small stationary device that can be distributed throughout an event site or venue and have a wired or wireless connection to a system database and/or data processor. Each kiosk has information about its specific location within the event site or venue, as well as access to information about that event/venue.

As illustrated in FIG. 2, there is third party system integration via the kiosk 22 for the event/parking ticketing entity, the point-of-sale payment processing, and social media outlets and the like. Data is then programmed to a wristband per proprietary protocol of an RFID locker manufacturer. One or more wristbands are then dispensed 18a having an RFID inlay and UID number or code which is linked to the patron and the point-of-sale initialization data. The kiosk software programs RFID wristband with the appropriate access control privileges per the lock or access control systems proprietary protocol (via a hard-coded script or in some cases network to a dedicated server). Once again, this may be saved in memory within the RFID wristband, and/or printed on the wristband in human readable and/or machine readable form.

As described above, in a particularly preferred embodiment, the patron orders the services online prior to their visit to the venue. A human readable or machine readable code printed on a receipt can be scanned to identify the patron. An RFID wristband is then encoded with a unique identifier that links to a database listing the purchased services. In an alternate embodiment, the list of services that have been ordered is encoded directly on the RFID wristband. As described and illustrated above, the wristbands may be mailed to the patron prior to the event, placed at will-call, or printed and created via a kiosk at the event or venue prior to admission.

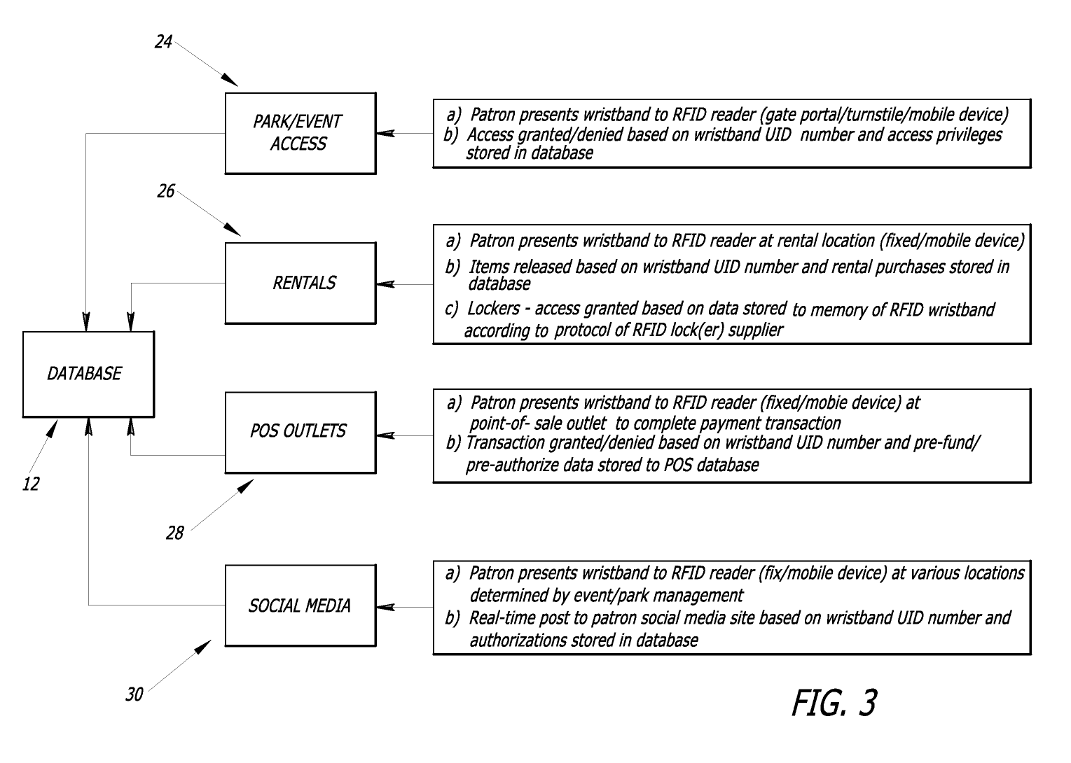

With reference now to FIG. 3, during their stay in the event site or venue, the patron utilizes the ordered services causing the database 12 and/or the wristband to be updated to reflect the consumed products or services. In one embodiment of the invention, the patron may also utilize a kiosk 22 to purchase additional services of products or services during their stay. At the end of the event reservation, the wristband can be scanned at the kiosk or a check-out station to determine if additional charges are due.

With continuing reference to FIG. 3, when entering the parking lot or event site/venue 24 the patron presents the transaction receipt and/or wristband to a reader in order to gain access to parking or the event itself. The reader is preferably housed in a kiosk or similar device located at entrances of the parking lot, event site/venue, or attractions, or at point-of-sale locations. Typically, the patron will have the wristband in his or her possession and placed on his or her wrist before entering the event or venue, and thus the patron presents the wristband to a RFID reader at the gate portal, turnstile, a mobile device, or the like. Access is granted or denied based upon the wristband UID number and access privileges stored in the database 12. If the unique identification code of the wristband corresponds with the patron ID file and purchased services, the patron is granted access.

With respect to rentals 26, the patron presents the wristband to an RFID reader at the rental location, which may be fixed or mobile. Items are released based upon the wristband UID number and rental purchases stored within the database. Similarly, lockers are granted access based upon the data stored to memory in the RFID wristband or the database according to the protocol of the RFID locker supplier.

With respect to point-of-sale outlets 28, additional services, concessions, etc., the patron presents the wristband to the RFID reader (which may be a fixed or mobile device) at the point-of-sale outlet to complete a payment transaction. The transaction is granted or denied based upon the wristband UID number/code and a pre-fund or pre-authorized data stored within the POS database or on the wristband itself.

In addition to the services identified above, the identity of the wearer may be linked via the wristband to any number of social media websites 30 or the like. Typically, the patron enters their social media user identification and password into the web portal or kiosk interface and accepts the site's terms and conditions. The user data is pushed directly to the secured log-in page of the social media site and the social media site may return a unique identifier back to the local database which is then linked to the individual's RFID wristband or the UID number of the RFID inlay in the wristband, whether manufactured before the event or dispensed from the kiosk. When the wristband is scanned at various locations throughout the event site/venue, the UID number of the wristband is read and the system software pushes the post to the specific user's social media account page. With continuing reference to FIG. 3, the patron presents the wristband to an RFID reader at one or more locations within the event or venue, as determined by the event/park management. Real-time posts to the patron's selected social media sites, based upon the wristband UID number and authorization stored in the database 12 occur.

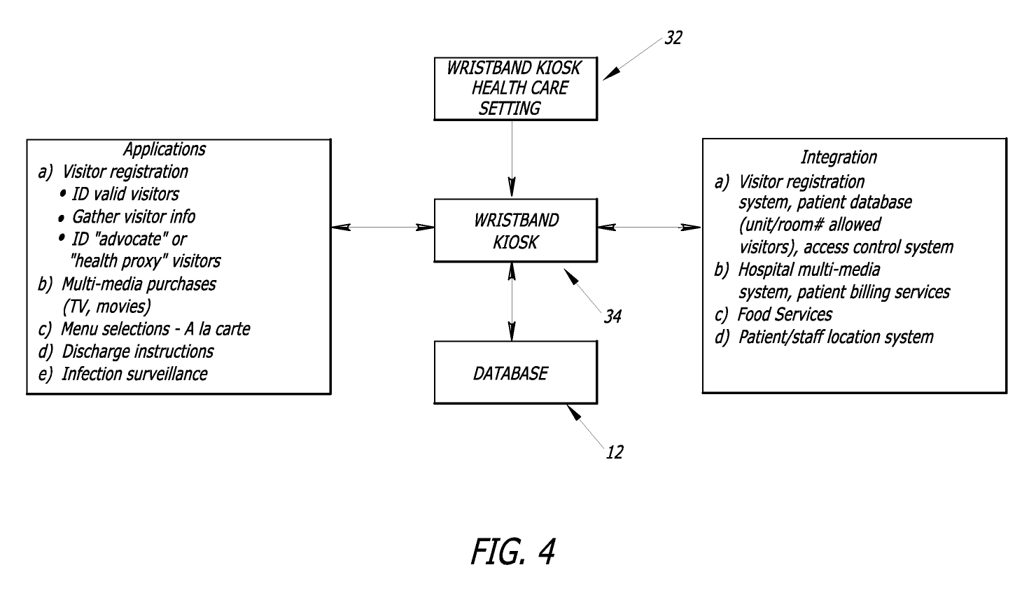

With reference now to FIG. 4, while the present invention has been described used in connection with an event site/venue, it will be understood that the present invention can be used in other situations, such as hospital visits, if the “patron” is an inmate to be processed in a jail facility, or any other situation where such a pre-admission process takes place. FIG. 4 illustrates the use of the wristband kiosk system of the present invention in connection with a healthcare setting 32. For example, while at the healthcare facility, such as a hospital, a wristband kiosk 34 may be accessed and used for different applications. These include visitor registration, including identification of valid visitors, gathering visitor information, and identifying whether the visitor is an “advocate” or “health proxy”. The visitor or patient may use the kiosk for multi-media purchases, such as television access, movies, games, etc. The menu selections can be customized by the healthcare facility. The kiosk may also be used for discharge instructions for the patient, infection surveillance, etc.

The kiosk 34, and a corresponding database 12, are used to integrate visitor registration, patient database (such as unit/room number, allowed visitors, etc.) and an access control system. The hospital multimedia system and patient billing service is also integrated with the system. Moreover, food services, patient/staff location systems and the like are all integrated in connection with the kiosk/database system of the present invention.

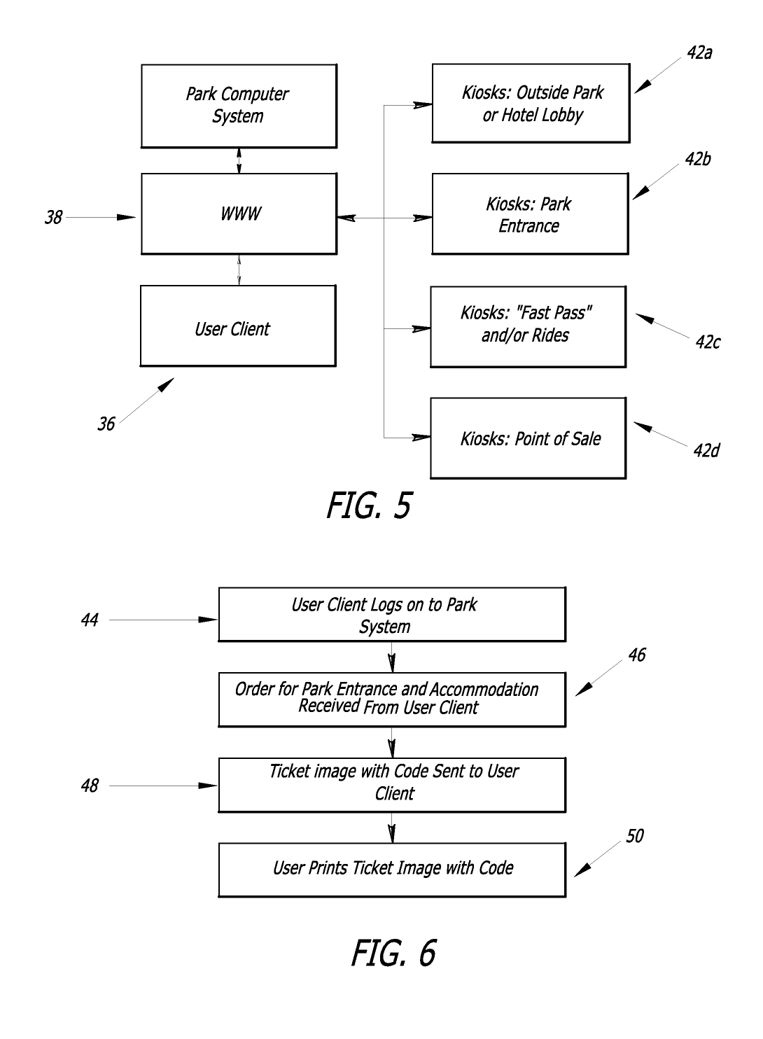

FIG. 5 illustrates in block diagram form the relationship and interaction of the various computer components of the system. The user client 36 component refers to a personal computer, tablet or smart phone through which a user may connect to the Internet 38 by wired connection or wirelessly. With this Internet connection 38, a user may access the park computer system 40 to perform all of the pre-admission activities discussed above. A plurality of types of kiosks 42a, 42b, 42c, and 42d at the event site, i.e., amusement park, also have access to the park computer system 40 through the Internet or an internal network 38, either by wired connection or wirelessly. The kiosks are located either outside the event site 42a, at the event entrance 42b, at the entrance to attractions 42c, or at point-of-sale locations 42d. The kiosks 42a, 42b, 42c, and 42d are available to perform the described functions, such as, dispensing identification articles, communicating availability of admission, permitting admission to the event/park or attractions, and completing point-of-sale transactions.

FIG. 6 illustrates in flowchart form how a user makes online reservations through the web portal. Through the user client over the Internet, a user logs into the park computer system 44. The user is then able to place an order for an event reservation 46. The event reservation may include park admission, lodging accommodations, and other benefits described above. The user receives a receipt or similar documentation, i.e., paper printout or electronic message, bearing a transaction code and/or patron ID 48. The patron ID corresponds to the user and identifies that user with a particular client log in. The transaction code corresponds to the event reservation and every service or benefit the user requested in the order. According to FIG. 6 the user can print the receipt bearing the transaction code and/or patron ID 50. The printed code may be in the form of a machine readable code such as a 2D (two dimensional) barcode or QR (quick response) code. The user may also carry the electronic message, i.e., e-mail or SMS message, on a tablet or smart phone or similar mobile device.

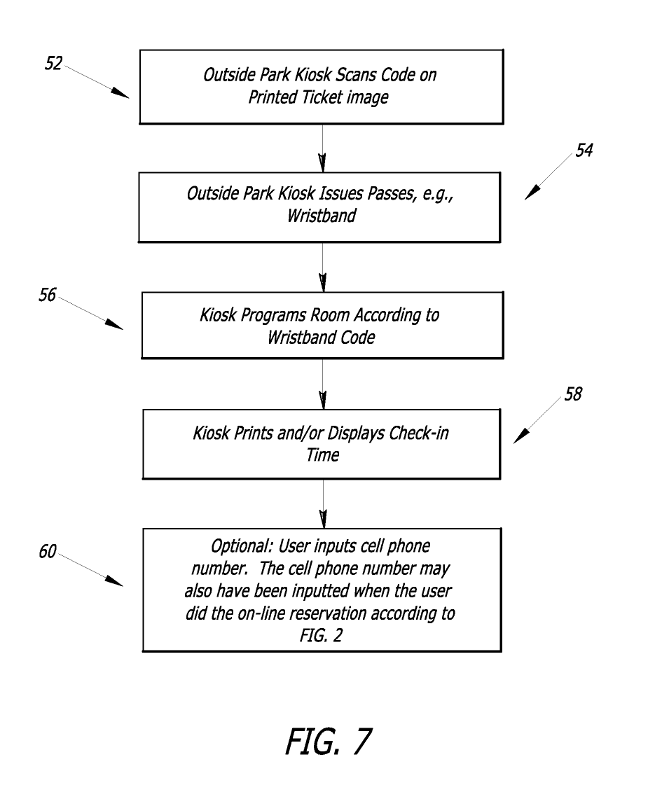

FIG. 7 illustrates in flowchart form the process of the inventive system issuing identification articles and notifies user when the event reservation, i.e., room accommodations are available. At a kiosk outside of the event site, the user scans a printed ticket image or other reservation paper bearing the patron ID or transaction code 52. According to an exemplary embodiment this includes scanning a machine readable code such as a 2D barcode. The user may also or alternatively type in a code number and/or provide a credit card that identifies the particular user. The code number or credit card identifying a particular user will correspond to the patron ID or transaction code discussed above.

In response to receiving an authentic patron ID or transaction code, the kiosk issues an identification article or ticket 54, such as, a wristband, a card or a label. Such identification article carries a machine readable code, preferably in the form of a barcode or an RFID chip. The barcode may be a 1-dimensional barcode, i.e., UPC barcode, or a 2-dimensional matrix barcode, i.e., a QR code. Through the system database and data processor, the kiosk associates the room accommodations with the machine readable code. In a particularly preferred embodiment, the system programs the room accommodations to respond to the machine readable code for entry and other related services 56.

The kiosk prints, displays or otherwise communicates to the user information about the room accommodations, i.e., location and availability 58. The user may optionally provide a telephone, i.e., mobile or cell phone, number by which the system, through the kiosk, database and/or data processor, may communicate information about the room accommodations 60.

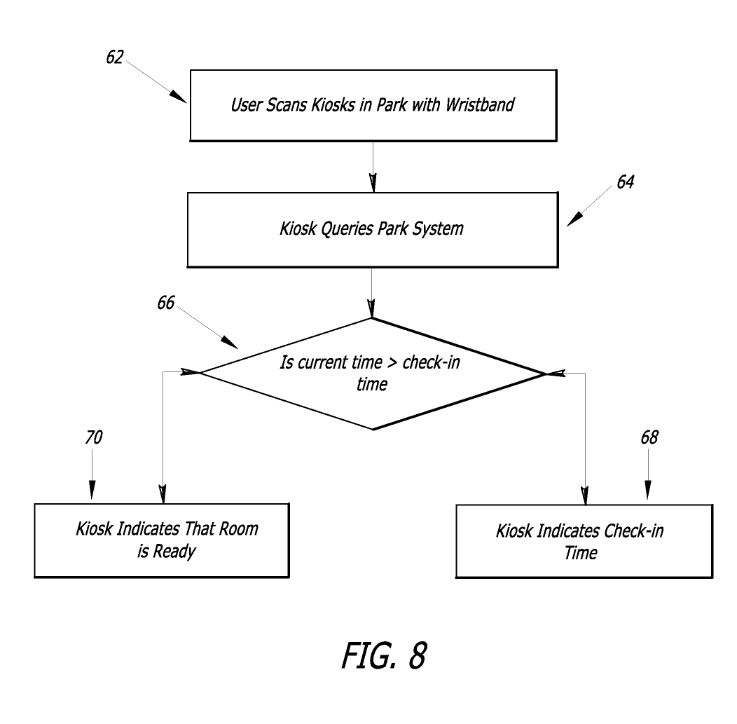

FIG. 8 illustrates in flowchart form how the system at the event site communicates with the user. Once inside the event site, i.e., amusement park, the user scans the machine readable code from the identification article at a kiosk 62. Using the transaction code, patron ID, or other information obtained from reading the machine readable code, the kiosk inside the park queries the database and/or data processor 64 to determine what benefits and/or privileges are associated with the information.

In the case of room accommodations, the system determines whether the current time corresponds to the check-in time 66. If the current time does not correspond to the check-in time, the kiosk communicates the proper check-in time 68. If the current time does correspond to the check-in time, the kiosk communicates that the room is ready 70. Such communication may be by display on a screen, print-out or text message/SMS to a telephone number provided previously. The system may stagger check-in times or room availability to minimize hallway and lobby congestion from a large number of guests arriving at the same time.

Although several embodiments have been described in detail for purposes of illustration, various modifications may be made without departing from the scope and spirit of the invention.

What is claimed is:

1. A computer based method performed by an event reservation system comprising a web portal, a kiosk and at least one event scanner, comprising the steps of:
through the web portal:
receiving an order from a user for an event reservation;
associating a transaction code with the order and a patron ID with the user; and
providing the transaction code and patron ID to the user;
at the kiosk:
receiving the transaction code or patron ID from the user; and
dispensing an identification article bearing a machine readable code corresponding to the transaction code or patron ID;
at a site of the event reservation:
reading the machine readable code from the identification article; and
permitting the user benefits of the event reservation in response to reading the machine readable code.

2. The computer based method of claim 1, wherein the event reservation comprises room accommodations, further comprising the steps of:
determining whether a current time has reached a check-in time for the room accommodations; and
providing availability and location information for the room accommodations in response to reading the machine readable code if the current time has reached the check-in time.

3. The computer based method of claim 1, further comprising the steps of:
receiving discretionary monetary funds from the user;
establishing an electronic spending account associated with the transaction code or patron ID and containing the discretionary monetary funds; and
permitting the user to make purchases with the discretionary monetary funds in the electronic spending account in response to reading the machine readable code.

4. The computer based method of claim 1, further comprising the steps of:
offering to the user through the web portal a registration page for a social media website account;
receiving account information from the user corresponding to the social media website account; and
associating the social media website account with the machine readable code.

5. The computer based method of claim 4, further comprising the steps of:
associating the order with the social media website account of the user; and
pushing an information post to the social media website account in response to reading the machine readable code.

6. The computer based method of claim 4, further comprising the steps of:
offering a social media website link to the user in response to the step of receiving the order; and
receiving a response from the user that authorizes pushing information posts to the social media website account.

7. An event reservation process, comprising the steps of:
receiving an order for an event reservation from a user through a web portal into an electronic database;
processing the order to associate a transaction code with the order and a patron ID with the user;
providing said transaction code and patron ID to the user through the web portal;
inputting the transaction code or patron ID from the user at a kiosk which then dispenses an identification article bearing a machine readable code associated with the event reservation ordered by the user through the web portal;
at the site of the event reservation, reading the machine readable code from the identification article; and
permitting the user to redeem the order received through the web portal.

8. The event reservation process of claim 7, wherein the event reservation comprises room accommodations, further comprising the steps of:
determining whether a current time has reached a check-in time for the room accommodations; and
providing availability and location information for the room accommodations in response to reading the machine readable code at the site of the event reservation if the current time has reached the check-in time.

9. The event reservation process of claim 7, further comprising the steps of:
receiving discretionary monetary funds from the user;
establishing an electronic spending account associated with the transaction code or patron ID and containing the discretionary monetary funds; and
permitting the user to make purchases with the discretionary monetary funds in the electronic spending account in response to reading the machine readable code at the site of the event reservation.

10. The event reservation process of claim 7, further comprising the steps of:
offering to the user through the web portal a registration page for a social media website account;
receiving account information from the user corresponding to the social media website account; and
associating the social media website account with the machine readable code.

11. The event reservation process of claim 10, further comprising the steps of:
associating the order with the social media website account of the user; and
pushing an information post to the social media website account in response to reading the machine readable code at the site of the event reservation.

12. The event reservation process of claim 10, further comprising the steps of:
offering a social media website link to the user in response to the step of receiving the order; and
receiving a response from the user that authorizes pushing information posts to the social media website account.

13. An event reservation and redemption process, comprising the steps of:
inputting an order for an event reservation from a user into a database;
associating a transaction code with the order and a patron ID with the user;
providing the transaction code and/or patron ID to the user;
dispensing an identification article bearing a machine readable code corresponding to the transaction code or patron ID; and
electronically reading the machine readable code from the identification article to allow the user to utilize the benefits of the event reservation.

14. The event reservation and redemption process of claim 13, wherein the event reservation comprises room accommodations, further comprising the steps of:
determining whether a current time has reached a check-in time for the room accommodations; and
providing availability and location information for the room accommodations in response to electronically reading the machine readable code at a site of the event reservation if the current time has reached the check-in time.

15. The event reservation and redemption process of claim 13, further comprising the steps of:
receiving discretionary monetary funds from the user;
establishing an electronic spending account associated with the transaction code or patron ID and containing the discretionary monetary funds; and
permitting the user to make purchases with the discretionary monetary funds in the electronic spending account in response to electronically reading the machine readable code at a site of the event reservation.

16. The event reservation and redemption process of claim 13, further comprising the steps of:
offering to the user a registration page for a social media website account;
receiving account information from the user corresponding to the social media website account; and
associating the social media website account with the machine readable code.

17. The event reservation and redemption process of claim 16, further comprising the steps of:
associating the order with the social media website account of the user; and
pushing an information post to the social media website account in response to electronically reading the machine readable code at a site of the event reservation.

18. An event reservation system, comprising:
a database and data processor configured for receiving an order from a user for an event reservation, associating a transaction code with the order, and associating a patron ID with the user;
a kiosk in electronic communication with the database and data processor, the kiosk configured to receive the transaction code or patron ID from the user and to dispense an identification article bearing a machine readable code corresponding to the transaction code or patron ID; and
an event machine in electronic communication with the database and data processor, the event machine configured to read the machine readable code from the identification article and to permit the user to exercise benefits associated with the event reservation.

19. The event reservation system of claim 18, wherein the event reservation comprises room accommodations, park admissions, parking reservations, locker rentals, stroller rentals, wheelchair rentals, and/or VIP privileges.

20. The event reservation system of claim 18, wherein the event reservation comprises room accommodations and the event machine is configured to determine whether a current time has reached a check-in time for the room accommodations, and to provide availability and location information for the room accommodations in response to reading the machine readable code if the current time has reached the check-in time.

21. The event reservation system of claim 18, further comprising an electronic spending account maintained in the database and in electronic communication with the data processor, the electronic spending account associated with the transaction code or the patron ID, the electronic spending account having a balance value commensurate with discretionary monetary funds deposited by the user, and the event machine configured to permit the user to make purchases with the discretionary monetary funds in the electronic spending account in response to reading the machine readable code.

22. The event reservation system of claim 18, further comprising a social media website link in the database, the social media website link configured to receive account information from the user corresponding to a social media website account of the user and to associate the social media website account with the machine readable code from the identification article.

23. The event reservation system of claim 22, wherein the social media website link is configured to associate the order with the social media website account of the user and to push an information post to the social media website account of the user in response to the event machine reading the machine readable code from the identification article.

24. The event reservation system of claim 18, wherein the identification article comprises a wristband, a label, an ID card, or a ticket bearing a bar code or an RFID chip.

25. The event reservation system of claim 24, wherein the wristband comprises an RFID inlay and/or a printed barcode linked to the transaction code, the patron ID and/or a pre-funded spending account.

26. The event reservation system of claim 18, wherein the order comprises patron information, purchase information, social media authorizations, and electronic spending account information.

27. The event reservation system of claim 26, wherein the patron information comprises name, address and payment information.

28. The event reservation system of claim 26, wherein the purchase information comprises admission tickets, rental items, VIP privileges, and parking passes.

29. The event reservation system of claim 28, wherein the rental items comprise lockers, strollers, and wheelchairs.

30. The event reservation system of claim 18, wherein the database and data processor are integrated with third-party systems providing event ticketing, point-of-sale/payment processing, social media providers, parking management, and/or rental locker management.

31. The event reservation system of claim 18, further comprising a transaction receipt provided to the user, wherein the transaction receipt contains the transaction code, the patron ID, a confirmation/transaction number and/or a purchase summary.

32. The event reservation system of claim 31, wherein the transaction code and/or patron ID corresponds to access rights, privileges, authorization and/or rentals of the user.

33. The event reservation system of claim 32, wherein the identification article is mailed to the user or placed on will-call at an event site.

34. The event reservation system of claim 18, wherein the kiosk is configured to receive the transaction code or patron ID by scanning a printed barcode or manual entry of the transaction code and/or patron ID via keypad or touchscreen.

35. The event reservation system of claim 18, wherein the kiosk is configured to receive a new order from the user for a new event reservation.

36. The event reservation system of claim 18, wherein the kiosk is positioned at an entry point to the event and is configured to grant or deny entry to or privileges of the event based upon the transaction code and/or patron ID when the user presents the identification article to an RFID reader.

37. The event reservation system of claim 36, wherein the kiosk comprises a gate, portal, turnstile, or mobile device.

38. The event reservation system of claim 18, wherein the kiosk is positioned at a rental location in the event and is configured to release rental items or permit access to lockers based upon the transaction code and/or patron ID when the user presents the identification article to an RFID reader.

39. The event reservation system of claim 38, wherein the kiosk comprises a fixed or mobile device.

40. The event reservation system of claim 18, wherein the kiosk is positioned at a point-of-sale outlet in the event and is configured to grant or deny a point-of-sale transaction based upon a pre-funded spending account and the transaction code and/or patron ID when the user presents the identification article to an RFID reader.

41. The event reservation system of claim 40, wherein the kiosk comprises a fixed or mobile device.

42. The event reservation system of claim 18, wherein a plurality of kiosks are positioned through the event and is configured to push an information post to the user's social media website account based upon the transaction code and/or patron ID when the user presents the identification article to an RFID reader.

43. The event reservation system of claim 42, wherein the kiosk comprises a fixed or mobile device.

44. The event reservation system of claim 18, wherein the event reservation corresponds to a healthcare appointment, multi-media purchases, menu selections, discharge instructions and/or infection surveillance.

45. The event reservation system of claim 44, wherein the multi-media purchases comprise television and/or movies.

46. The event reservation system of claim 44, wherein the database and data processor are integrated with third-party systems comprising a visitor registration system, a patient database, an access control system, a hospital multi-media system, a patient billing service, a food service, and/or a patient/staff location system.

47. The event reservation system of claim 18, wherein the user accesses the database and data processor over the internet through a personal computer, tablet computing device, or smart phone.
